(19) United States
(12) Patent Application Publication
(10) Pub. No.: US 2005/0059609 A1
(43) Pub. Date: Mar. 17, 2005

(54) New alpha crystalline form of perindopril tert-butylamine salt

(76) Inventors: Bruno Pfeiffer, Saint Leu La Foret (FR); Yves-Michel Ginot, Orleans (FR); Gerard Coquerel, Boos (FR); Stephane Beilles, Rouen (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/792,355

(22) Filed: Mar. 3, 2004

Related U.S. Application Data
(63) Continuation of application No. 10/312,961, filed on Dec. 31, 2002, now abandoned.

(30) Foreign Application Priority Data
Jul. 6, 2000 (FR) 00/08973
Jul. 6, 2001 (WO) PCT/FR01/02167
Jul. 6, 2000 (FR) 00.08793

(51) Int. Cl. A61K 38/04; C07D 209/42

(52) U.S. Cl. 514/19; 548/492

(57) ABSTRACT

An α crystalline form of the compound of formula (I): 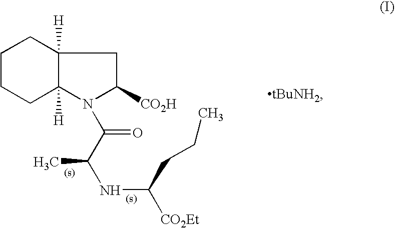 characterized by its powder X-ray diffraction data. Medicinal products containing the same which are useful as inhibitors of angiotensin I converting enzyme.

The present invention relates to a new α crystalline form of perindopril tert-butylamine salt of formula (I): 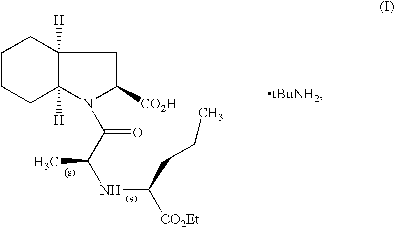
to a process for its preparation and to pharmaceutical compositions containing it.

Perindopril and its pharmaceutically acceptable salts, and more especially its tert-butylamine salt, have valuable pharmacological properties.

Their principal property is that of inhibiting angiotensin I converting enzyme (or kininase II), which prevents, on the one hand, conversion of the decapeptide angiotensin I to the octapeptide angiotensin II (a vasoconstrictor) and, on the other hand, degradation of bradykinin (a vasodilator) to an inactive peptide.

Those two actions contribute to the beneficial effects of perindopril in cardiovascular diseases, more especially in arterial hypertension and heart failure.

Perindopril, its preparation and its use in therapeutics have been described in European Patent specification EP 0 049 658.

In view of the pharmaceutical value of this compound, it has been of prime importance to obtain it with excellent purity. It has also been important to be able to synthesise it by means of a process that can readily be converted to the industrial scale, especially in a form that allows rapid filtration and drying. Finally, that form had to be perfectly reproducible, easily formulated and sufficiently stable to allow its storage for long periods without particular requirements for temperature, light, humidity or oxygen level.

The patent specification EP 0 308 341 describes an industrial synthesis process for perindopril. However, that document does not specify the conditions for obtaining perindopril in a form that exhibits those characteristics in a reproducible manner.

The Applicant has now found that a particular salt of perindopril, the tert-butylamine salt, can be obtained in a well defined, perfectly reproducible crystalline form that especially exhibits valuable characteristics of filtration, drying and ease of formulation.

More specifically, the present invention relates to the a crystalline form of the compound of formula (I), characterised by the following powder X-ray diffraction diagram, measured using a Siemens D5005 diffractometer (copper anticathode) and expressed in terms of inter-planar distance d, Bragg's angle 2 theta, intensity and relative intensity (expressed as a percentage of the most intense ray):

| | | | Relative |
|---|---|---|---|
| Angle 2 | Inter-planar | | intensity |
| theta (°) | distance d (Å) | Intensity | (%) |
| 7.680 | 11.50 | 390 | 8.8 |
| 8.144 | 10.85 | 230 | 5.2 |
| 9.037 | 9.78 | 4410 | 100 |
| 10.947 | 8.08 | 182 | 4.1 |
| 13.150 | 6.73 | 82 | 1.9 |
| 13.687 | 6.46 | 83 | 1.9 |
| 14.627 | 6.05 | 582 | 13.2 |
| 15.412 | 5.74 | 770 | 17.5 |
| 16.573 | 5.34 | 1115 | 25.3 |
| 17.357 | 5.10 | 340 | 7.7 |
| 18.109 | 4.89 | 193 | 4.4 |
| 19.922 | 4.45 | 306 | 6.9 |
| 20.609 | 4.31 | 375 | 8.5 |
| 21.412 | 4.15 | 226 | 5.1 |
| 21.832 | 4.07 | 217 | 4.9 |
| 22.158 | 4.01 | 483 | 11 |
| 22.588 | 3.93 | 386 | 8.8 |
| 23.323 | 3.81 | 107 | 2.4 |
| 24.200 | 3.67 | 448 | 10.2 |
| 24.727 | 3.60 | 137 | 3.1 |
| 25.957 | 3.43 | 125 | 2.8 |
| 26.932 | 3.31 | 75 | 1.7 |
| 27.836 | 3.20 | 197 | 4.5 |
| 28.966 | 3.08 | 129 | 2.9 |
| 29.213 | 3.05 | 117 | 2.7 |

The invention relates also to a process for the preparation of the α crystalline form of the compound of formula (I), which process is characterised in that a solution of perindopril tert-butylamine salt in ethyl acetate is heated at reflux and is cooled gradually until crystallisation is complete.
In the crystallisation process according to the invention it is possible to use the compound of formula (I) obtained by any process. Advantageously, the compound of formula (1) obtained by the preparation process described in patent specification EP 0 308 341 is used.
The concentration of the compound of formula (I) in the ethyl acetate is preferably from 70 to 90 g/litre.
Advantageously, the solution of the compound of formula (I) in ethyl acetate at reflux is first cooled to a temperature of from 55 to 65° C. at a rate of from 5 to 110° C./hour, preferably from 6 to 8° C./hour, and then to ambient temperature.
The solution can advantageously be seeded during the cooling step at a temperature of from 76 to 65° C.
The perindopril tert-butylamine salt that is thereby obtained is in the form of individual needles about 0.2 mm long. That homogeneous distribution has the advantage of allowing especially rapid and efficient filtration and drying, as well as allowing the preparation of pharmaceutical formulations having a uniform and reproducible composition, which is especially advantageous when those formulations are intended for oral administration.
The form thereby obtained is sufficiently stable to allow its storage for long periods without particular requirements for temperature, light, humidity or oxygen level.

The invention relates also to pharmaceutical compositions comprising as active ingredient the α crystalline form of the compound of formula (I) together with one or more appropriate, inert, non-toxic excipients. Among the pharmaceutical compositions according to the invention, there may be mentioned more especially those that are suitable for oral, parenteral (intravenous or subcutaneous) or nasal administration, tablets or dragees, sublingual tablets, gelatin capsules, lozenges, suppositories, creams, ointments, dermal gels, injectable preparations, drinkable suspensions etc.

The useful dosage can be varied according to the nature and severity of the disorder, the administration route and the age and weight of the patient. It varies from 1 to 500 mg per day in one or more administrations.

The pharmaceutical compositions according to the invention may also comprise a diuretic such as indapamide.

The following Examples illustrate the invention but do not limit it in any way.

The powder X-ray diffraction spectrum was measured under the following experimental conditions:
Siemens D5005 diffractometer, scintillation detector,
copper anticathode (γ=1.5405 Å), voltage 40 kV, intensity 40 mA,
mounting θ-θ,
measurement range: 5° to 30°,
increment between each measurement: 0.02°,
measurement time per step: 2 s,
variable slits v6,
filter Kβ (Ni),
no internal reference,
zeroing procedure using the Siemens slits,
experimental data processed using EVA software (version 5.0).

### EXAMPLE 1

### α Crystalline Form of Perindopril Tert-Butylamine Salt

125 g of perindopril tert-butylamine salt obtained according to the process described in patent specification EP 0 308 341 are dissolved in 1.68 litres of ethyl acetate heated at reflux.

The temperature of the solution is then brought to 60° C. in the course of 2 hours 30 minutes and is then cooled to ambient temperature.

The solid obtained is collected by filtration.

Powder X-Ray Diffraction Diagram:

The powder X-ray diffraction profile (diffraction angles) of the α form of perindopril tert-butylamine salt is given by the significant rays collated in the following table together with the intensity and relative intensity (expressed as a percentage of the most intense ray).

| | | | Relative |
|---|---|---|---|
| Angle 2 | Inter-planar | | intensity |
| theta (°) | distance d (Å) | Intensity | (%) |
| 7.680 | 11.50 | 390 | 8.8 |
| 8.144 | 10.85 | 230 | 5.2 |
| 9.037 | 9.78 | 4410 | 100 |
| 10.947 | 8.08 | 182 | 4.1 |
| 13.150 | 6.73 | 82 | 1.9 |
| 13.687 | 6.46 | 83 | 1.9 |
| 14.627 | 6.05 | 582 | 13.2 |
| 15.412 | 5.74 | 770 | 17.5 |
| 16.573 | 5.34 | 1115 | 25.3 |
| 17.357 | 5.10 | 340 | 7.7 |
| 18.109 | 4.89 | 193 | 4.4 |
| 19.922 | 4.45 | 306 | 6.9 |
| 20.609 | 4.31 | 375 | 8.5 |
| 21.412 | 4.15 | 226 | 5.1 |
| 21.832 | 4.07 | 217 | 4.9 |
| 22.158 | 4.01 | 483 | 11 |
| 22.588 | 3.93 | 386 | 8.8 |
| 23.323 | 3.81 | 107 | 2.4 |
| 24.200 | 3.67 | 448 | 10.2 |
| 24.727 | 3.60 | 137 | 3.1 |
| 25.957 | 3.43 | 125 | 2.8 |
| 26.932 | 3.31 | 75 | 1.7 |
| 27.836 | 3.20 | 197 | 4.5 |
| 28.966 | 3.08 | 129 | 2.9 |
| 29.213 | 3.05 | 117 | 2.7 |

### EXAMPLE 2

### Pharmaceutical Composition

Preparation formula for 1000 tablets each containing 4 mg of active ingredient:

| | |
|---|---|
| Compound of Example 1 | 4 g |
| Hydroxypropylcellulose | 2 g |
| Wheat starch | 10 g |
| Lactose | 100 g |
| Magnesium stearate | 3 g |
| Talc | 3 g |

1. α crystalline form of the compound of formula (I): 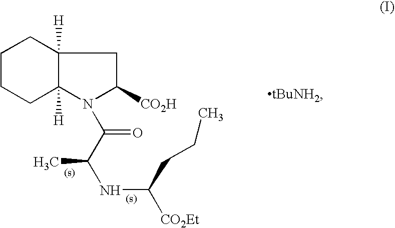
characterised by the following powder X-ray diffraction diagram, measured using a diffractometer (copper anticathode) and expressed in terms of inter-planar distances d, Bragg's angle 2 theta, intensity and relative intensity (expressed as a percentage with respect to the most intense ray):

| | | | Relative |
|---|---|---|---|
| Angle 2 | Inter-planar | | intensity |
| theta (°) | distance d (Å) | Intensity | (%) |
| 7.680 | 11.50 | 390 | 8.8 |
| 8.144 | 10.85 | 230 | 5.2 |
| 9.037 | 9.78 | 4410 | 100 |
| 10.947 | 8.08 | 182 | 4.1 |
| 13.150 | 6.73 | 82 | 1.9 |
| 13.687 | 6.46 | 83 | 1.9 |
| 14.627 | 6.05 | 582 | 13.2 |
| 15.412 | 5.74 | 770 | 17.5 |
| 16.573 | 5.34 | 1115 | 25.3 |
| 17.357 | 5.10 | 340 | 7.7 |
| 18.109 | 4.89 | 193 | 4.4 |
| 19.922 | 4.45 | 306 | 6.9 |
| 20.609 | 4.31 | 375 | 8.5 |
| 21.412 | 4.15 | 226 | 5.1 |
| 21.832 | 4.07 | 217 | 4.9 |
| 22.158 | 4.01 | 483 | 11 |
| 22.588 | 3.93 | 386 | 8.8 |
| 23.323 | 3.81 | 107 | 2.4 |
| 24.200 | 3.67 | 448 | 10.2 |
| 24.727 | 3.60 | 137 | 3.1 |
| 25.957 | 3.43 | 125 | 2.8 |
| 26.932 | 3.31 | 75 | 1.7 |
| 27.836 | 3.20 | 197 | 4.5 |
| 28.966 | 3.08 | 129 | 2.9 |
| 29.213 | 3.05 | 117 | 2.7 |

2. Process for the preparation of the α crystalline form of the compound of formula (I) according to claim 1, characterised in that a solution of perindopril tert-butylamine salt in ethyl acetate is heated at reflux and is then cooled gradually until crystallisation is complete.

3. Process according to claim 2, characterised in that the compound of formula (I) obtained by the preparation process described in patent specification EP 0 308 341 is used.

4. Process according to either claim 2 or claim 3, characterised in that the concentration of the compound of formula (I) in the ethyl acetate is from 70 to 90 g/litre.

5. Process according to any one of claims 2 to 4, characterised in that the solution of the compound of formula (I) in ethyl acetate at reflux is first cooled to a temperature of from 55 to 65° C. at a rate of from 5 to 10° C./hour, and then to ambient temperature.

6. Process according to any one of claims 2 to 4, characterised in that the solution of the compound of formula I in ethyl acetate is seeded during the cooling step at a temperature of from 76 to 65° C.

7. Process according to claim 5, characterised in that the solution of the compound of formula (I) in ethyl acetate at reflux is first cooled to a temperature of from 55 to 65° C. at a rate of from 6 to 8° C./hour, and then to ambient temperature.

8. Process according to any one of claims 2 to 7, characterised in that the perindopril tert-butylamine salt that is thereby obtained is in the form of readily filterable individual needles.

9. Pharmaceutical composition comprising as active ingredient the compound according to claim 1, in combination with one or more pharmaceutically acceptable, inert, non-toxic carriers.

10. Pharmaceutical composition according to claim 9 for use in the manufacture of medicaments for use as inhibitors of angiotensin I converting enzyme.

11. Pharmaceutical composition according to claim 10 for use in the manufacture of medicaments for use in the treatment of cardiovascular diseases.

12. Pharmaceutical composition according to any one of claims 9 to 11, characterised in that it also comprises a diuretic.

13. Pharmaceutical composition according to claim 12, characterised in that the diuretic is indapamide.

14. An α crystalline form of the compound of formula (I): 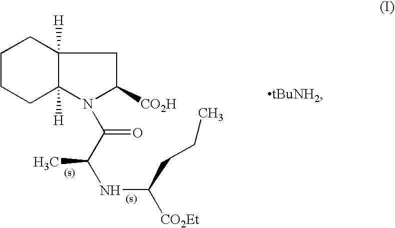
exhibiting essentially the following powder X-ray diffraction data, measured using a diffractometer (copper anticathode) and expressed in terms of inter-planar distances d, Bragg's angle 2 theta, intensity and relative intensity (expressed as a percentage with respect to the most intense ray)

| | | | Relative |
|---|---|---|---|
| Angle 2 | Inter-planar | | intensity |
| theta (°) | distance d (Å) | Intensity | (%) |
| 7.680 | 11.50 | 390 | 8.8 |
| 8.144 | 10.85 | 230 | 5.2 |
| 9.037 | 9.78 | 4410 | 100 |
| 10.947 | 8.08 | 182 | 4.1 |
| 13.150 | 6.73 | 82 | 1.9 |
| 13.687 | 6.46 | 83 | 1.9 |
| 14.627 | 6.05 | 582 | 13.2 |
| 15.412 | 5.74 | 770 | 17.5 |
| 16.573 | 5.34 | 1115 | 25.3 |
| 17.357 | 5.10 | 340 | 7.7 |
| 18.109 | 4.89 | 193 | 4.4 |
| 19.922 | 4.45 | 306 | 6.9 |
| 20.609 | 4.31 | 375 | 8.5 |
| 21.412 | 4.15 | 226 | 5.1 |
| 21.832 | 4.07 | 217 | 4.9 |
| 22.158 | 4.01 | 483 | 11 |
| 22.588 | 3.93 | 386 | 8.8 |
| 23.323 | 3.81 | 107 | 2.4 |
| 24.200 | 3.67 | 448 | 10.2 |
| 24.727 | 3.60 | 137 | 3.1 |
| 25.957 | 3.43 | 125 | 2.8 |
| 26.932 | 3.31 | 75 | 1.7 |
| 27.836 | 3.20 | 197 | 4.5 |
| 28.966 | 3.08 | 129 | 2.9 |
| 29.213 | 3.05 | 117 | 2.7 |

15. A process for the preparation of the α crystalline form of the compound of claim 14, wherein a solution of perindopril tert-butylamine salt in ethyl acetate is heated at reflux and is then cooled gradually until crystallization is complete.

16. The process of claim 15, wherein the compound of formula (I) obtained by the preparation process described in patent specification EP 0 308 341 is used.

17. The process of claim 15, wherein the concentration of the compound of formula (I) in the ethyl acetate is 70 to 90 g/litre.

18. The process of claim 15, wherein the solution of the compound of formula (I) in ethyl acetate at reflux is first cooled to a temperature of 55 to 65° C. at a rate of 5 to 10° C./hour, and then to ambient temperature.

19. The process of claim 15, wherein the solution of the compound of formula (I) in ethyl acetate is seeded during the cooling step at a temperature of 65 to 76° C.

20. The process of claim 18, wherein the solution of the compound of formula (I) in ethyl acetate at reflux is first cooled to a temperature of 55 to 65° C. at a rate of 6 to 8° C./hour, and then to ambient temperature.

21. The process of claim 15, wherein the perindopril tert-butylamine salt thereby obtained is in the form of readily filterable individual needles.

22. A method of treating a living animal body afflicted with a condition requiring an inhibitor of angiotensin I converting enzyme, comprising the step of administering to the living animal body an amount of the compound of claim 14 which is effective for alleviation of the condition.

23. A pharmaceutical composition comprising, as active principle, an effective amount of the compound of claim 14, together with one or more pharmaceutically acceptable excipients or vehicles.

24. A method of treating a living animal body afflicted with a cardiovascular disease, comprising the step of administering to the living animal body an amount of the compound of claim 14 which is effective for alleviation of the condition.

25. The pharmaceutical composition of claim 23, which also comprises a diuretic.

26. The pharmaceutical composition of claim 25, wherein the diuretic is indapamide.
